(19) United States
(12) Patent Application Publication
(10) Pub. No.: US 2008/0213874 A1
(43) Pub. Date: Sep. 4, 2008

(54) WEIGHT MEASUREMENTS OF LIQUIDS IN FLEXIBLE CONTAINERS

(75) Inventors: Peter A. Mitchell, East Greenwich, RI (US); Michael Fisher, Ashland, MA (US)

Correspondence Address: WOLF GREENFIELD & SACKS, P.C., 600 ATLANTIC AVENUE, BOSTON, MA, 02210-2206, US

(73) Assignee: Xcellerex, Inc., Marlborough, MA (US)

(21) Appl. No.: 12/039,052

(22) Filed: Feb. 28, 2008

Related U.S. Application Data
(60) Provisional application No. 60/903,977, filed on Feb. 28, 2007.

(51) Int. Cl. C12M 1/34; G01F 22/00

(52) U.S. Cl. 435/287.1; 73/149

(57) ABSTRACT

Articles and methods for measuring weight of a liquid in a disposable bag bioreactor are presented. In certain embodiments, bioreactor systems described herein include a supported container (e.g., a flexible bag in a reusable housing) for containing a liquid, and at least two pressure indicating sensors operatively associated with the container. A first pressure indicating sensor can be placed near the bottom of the container to measure the total downward force within the container, including the liquid head in the container and the gas pressure above the liquid. The second pressure indicating sensor can be placed near the top of the container to measure only the pressure at the top of or above the liquid. Signals from the pressure indicating sensors can be directed to a control system that receives the signals and calculates a difference between the signals. This difference can be used to determine a volume or a weight of the liquid in the container. Advantageously, real-time weight measurements can be obtained while the system is in operation and continuous flow processes can be monitored. Moreover, in some embodiments, the pressure indicating sensors are isolated from contact with any fluid (e.g., liquid) in the container and, therefore, do not require cleaning after processing of each batch of reactants. Contamination of the process fluid by contact with the pressure indicating sensors can also be avoided.

### RELATED APPLICATIONS

This application claims priority under 35 U.S.C. 119(e) to U.S. Provisional Patent Application No. 60/903,977, filed Feb. 28, 2007, entitled “Weight Measurements of Liquids in Flexible Containers”, which is incorporated herein by reference in its entirety.

### FIELD OF INVENTION

The present invention relates generally to flexible containers, and more specifically, to systems and methods for measuring weight of a liquid contained in a flexible container, such as a disposable bag, supported by a reusable support structure. The flexible containers may be used as bioreactors for performing chemical and/or biological reactions contained therein.

### BACKGROUND

Systems and techniques for determining the weight and/or volume of liquid contained in bioreactors including flexible containers, such as disposable bags contained in reusable support structures, have been designed to include pressure indicating sensors for measuring weight and/or volume of a liquid in the bioreactor are known. Such systems and techniques include floor scales or single pressure or force transducers configured to be in fluid communication with process liquid. The former systems have disadvantages of requiring cumbersome, large and often costly scales and to being susceptible to vibration and shock. The later systems suffer from being inaccurate for applications where there may be a gas phase present in the flexible container at greater than atmospheric pressure and from having the potential to introduce contamination into the process fluid. In the context of rigid reactor vessels not including flexible containers/liners, such as disposable bags, systems have been designed to include pressure indicating sensors for measuring weight and/or volume of a liquid in the reactor. However such systems are not well suited for measuring weight and/or volume of a liquid in a flexible container, such as a disposable bag. Accordingly, a system including one or more pressure or force indicating sensors that could facilitate measurement of the weight and/or volume of a liquid in a flexible container of a bioreactor system, where the pressure indicating sensors can be reused without being cleaned after use, and where at least some of the above-indicated disadvantages of typical conventional systems for determining weight and/or volume are reduced or avoided would be beneficial.

### SUMMARY OF THE INVENTION

Systems and methods for determining at least one parameter indicative of the weight of a liquid contained in a flexible container, such as a disposable bag, in a bioreactor support structure, are described.

In one aspect of the invention, a series of bioreactor systems are provided. In one embodiment, a bioreactor system comprises a flexible container for housing a liquid in the container and a reusable support structure for surrounding and containing the flexible container. The bioreactor system also includes a first pressure or force indicating sensor operatively associated with the flexible container but not in fluid contact with any fluid in the flexible container, the pressure or force indicating sensor configured to measure a parameter indicative of a first pressure in the flexible container. The bioreactor system also includes a second pressure or force indicating sensor operatively associated with the flexible container, the pressure or force indicating sensor configured to measure a parameter indicative of a second pressure in the flexible container. A control system may be configured to receive input from the first and second pressure or force indicating sensors indicative of the first and second pressures and to calculate a difference between the first and second pressures.

In another embodiment, a bioreactor system comprises a flexible container for housing a liquid in the container and a reusable support structure for surrounding and containing the flexible container. The bioreactor system may also include a first pressure or force indicating sensor operatively associated with the flexible container and configured to measure a parameter indicative of a first pressure in the flexible container and a second pressure or force indicating sensor operatively associated with the flexible container and configured to measure a parameter indicative of a second pressure in the flexible container. The bioreactor system may also include a control system configured to receive input from the first and second pressure or force indicating sensors indicative of the first and second pressures and to calculate a difference between the first and second pressures.

In another embodiment, a bioreactor system comprises a flexible container for housing a liquid in the container, at least one pressure or force indicating sensor operatively associated with the flexible container but not in contact with any fluid in the flexible container, the pressure or force indicating sensor configured to measure a parameter indicative of a pressure in the flexible container. The bioreactor system may also include a control system configured to receive input from the at least one pressure or force indicating sensor indicative of the pressure, the control system configured to determine from the input a volume or a weight of a liquid in the flexible container.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic diagram of a flexible container comprising a disposable bag surrounded by a reusable support structure and including pressure indicating transmitters according to one embodiment of the invention;

FIG. 2 shows a pressure indicating transmitter that is positioned flush against a flexible container according to one embodiment of the invention;

FIG. 3 shows a pressure indicating transmitter that is positioned at the bottom of a reusable support structure according to one embodiment of the invention;

FIG. 4 shows a pressure indicating transmitter including a gauge guard with an integral molded fitting diaphragm according to one embodiment of the invention;

FIG. 5 shows a pressure indicating transmitter including a gauge guard, in cross-section, with a sealed diaphragm unit according to one embodiment of the invention;

FIG. 6 shows a bioreactor system comprising a disposable bioreactor bag and a reusable support structure in connection with a variety of components for carrying out a chemical and/or biological reaction inside the bag according to one embodiment of the invention; and

FIG. 7 shows a plot illustrating measurements of pressure in a flexible container using pressure indicating transmitters described herein.

### DETAILED DESCRIPTION

The present invention relates generally to bioreactors, and more specifically, to articles and methods for measuring weight of a liquid in a flexible container of a bioreactor. “Flexible container” or “flexible bag” as used herein, indicates that the container/bag is unable to maintain its shape and/or structural integrity when subjected to the internal pressures (e.g., due to the weight and/or hydrostatic pressure of liquids and/or gases contained therein expected during operation without the benefit of a separate support structure. The flexible container may be made out of inherently flexible materials, such as many plastics, or may be made out of what are normally considered rigid materials (e.g., glass or certain metals) but having a thickness and/or physical properties rendering the container as a whole unable to maintain its shape and/or structural integrity when subjected to the internal pressures expected during operation without the benefit of a separate support structure.

Although much of the description herein involves an exemplary application of the present invention related to bioreactors, the invention and its uses are not so limited, and it should be understood that the invention can also be used to measure weight and/or volume of a liquid (and/or pressure of a liquid or gas inside a flexible container) in other settings. Such settings may include, for example, blow molding, bag manufacture, and other processes involving inflation of a flexible container and/or containment of a fluid in a flexible container.

In certain embodiments, bioreactor systems described herein include a disposable container (e.g., a flexible, disposable bag, which may be sterilized prior to use and may be designed for a single use) for housing a liquid and at least two pressure or force indicating sensors, such as pressure/force transducers, pressure/force transmitters, pressure/force gauges, or other pressure or force measuring device, operatively associated with the container (e.g., mechanically, electrically, or fluidically, such as by hoses or tubing, etc.). The pressure indicating sensors may be operatively associated with the container by, for example, being connected to the flexible container directly (e.g., to a wall of the flexible container) or by being fluidically connected to a component of the flexible container (e.g., via a hose or tubing that is connected to the flexible container). Attachment may occur reversibly or irreversibly. In certain embodiments, the pressure indicating sensors may be operatively associated with the container without being attached or connected thereto but by simply being located adjacent to and in contact with the flexible container. In general, as used herein, a component of an inventive system that is “operatively associated with” one or more other components indicates that such components are directly connected to each other, in direct physical contact with each other without being connected or attached to each other, or are not directly connected to each other or in contact with each other, but are mechanically, electrically (including via electromagnetic signals transmitted through space), or fluidically interconnected so as to cause or enable the components so associated to perform their intended functionality. Accordingly, the pressure indicating sensor may be operatively associated with the container by being positioned adjacent a flexible container and held in place by any suitable manner (e.g., by gravity, by use of an adhesive, etc.) so as to allow the pressure indicating sensor to perform its function.

In some embodiments, a first pressure indicating sensor may be placed at or near the bottom of the flexible container to measure the total downward force/pressure within the container, such measurement including the force/pressure due to the weight of the liquid in the container and also any gas pressure present above the liquid. The second pressure or force indicating sensor may be placed at or near the top of the container to measure only the pressure/force at the top of the container, for example in a gas-containing head space above the liquid. Input or data, for example in the form of electrical or electromagnetic signals from the pressure indicating sensors, can be directed to a control system, such as a computer implemented system that receives the input and calculates a difference between the signals. This difference indicates, and may be used to calculate, a volume or a weight of the liquid in the flexible container, as explained further below. Advantageously, continuous weight measurements can be obtained while the system is in operation and continuous flow processes can be monitored. In certain embodiments, such a control system may also perform other system measurement and/or control tasks, such as automated switching of an exhaust stream from a first vent filter to another if the first vent filter becomes partially or completely blocked. Moreover, in some embodiments, the pressure indicating sensors are not in contact with any fluid (e.g., liquid) in the flexible container and, therefore, do not require cleaning after processing of each batch of reactants and do not have the ability to cause contamination of the process fluid.

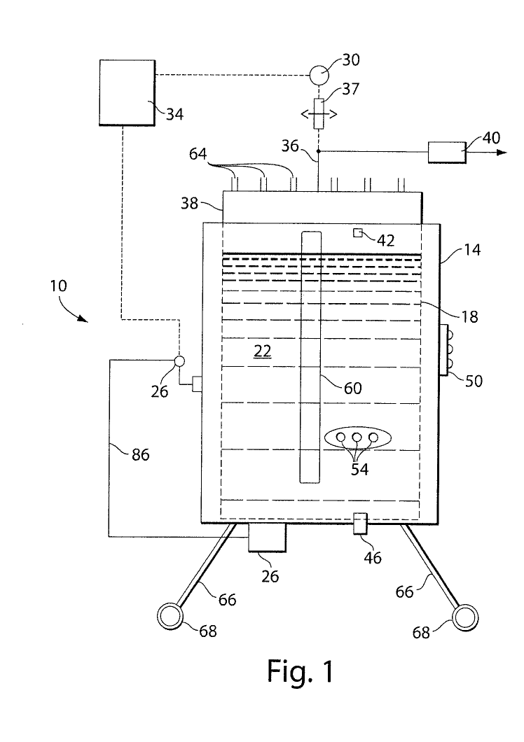

An example of a bioreactor system including pressure indicating sensors for measuring weight of a liquid in a flexible, disposable container is shown schematically in FIG. 1. As shown in the embodiment illustrated in FIG. 1, bioreactor system 10 includes a reusable support structure 14 (e.g., a stainless steel tank) that surrounds and contains a flexible container 18. In some embodiments, the flexible container is configured as a bag (e.g., a polymeric bag). Additionally and/or alternatively, all or portions of the flexible bag or other flexible container may comprise a substantially rigid material such as a rigid polymer, metal, and/or glass. The flexible container may be disposable and may be configured to be easily removable from the support structure. In some embodiments, the flexible container is non-integrally connected to the support structure. As used herein, the term “integrally connected,” when referring to two or more objects, means separation of the two or more objects requires causing damage to at least one of the object (or components of the object), for example, by breaking or peeling (e.g., separating components fastened together via adhesives, tools, etc.).

Flexible container 18 may be constructed and arranged for housing a liquid 22, which may contain reactants, media, and/or other components necessary for carrying out a chemical and/or biological reaction. Flexible container 18 may also be configured such that liquid 22 remains substantially in contact only with the flexible container during use and not in contact with reusable support structure 14. In such embodiments, the container may be disposable and used for a single reaction or a single series of reactions, after which the disposable container is discarded. Because the liquid in the disposable container may not come into contact with the support structure, the support structure can be reused without cleaning. That is, after a reaction takes place in container 18, the container can be removed from the support structure and replaced by a second disposable container. A second reaction can be carried out in the second disposable container without having to clean either the disposable container or the reusable support structure.

To measure the weight and/or volume of the liquid in the flexible container, first and second pressure indicating transmitters 26 and 30, respectively, may be operatively associated with the container and/or tank, e.g., by mechanical or electrical means, by direct physical contact with each other without being connected or attached to one another, or by being fluidically connected to the container and/or tank, such as by tubing. The pressure indicating transmitters may in certain embodiments comprise a measuring cell (e.g., a piezoelectric sensor) for sensing pressures or forces and electronic circuitry to process signals from the measuring cells and to send information to a control system 34. Accordingly, the first and second pressure indicating transmitters can be configured to measure first and second pressures in the flexible container, the difference indicating or usable to determine a volume or weight of the liquid in the container. Moreover, in some embodiments, the first and/or second pressure indicating transmitters are not in liquid contact with any liquid in the container. For instance, as shown in the illustrative embodiment of FIG. 1, first pressure indicating transmitter 26 is positioned at or near the bottom of the container (e.g., with a pressure or force measuring component positioned between the container and the support structure) such that when the container is filled with a liquid, the downward force/pressure of the liquid in the container is applied against the pressure indicating transmitter (without the liquid being in contact with the measuring cell). The magnitude of the force/pressure reflects the amount of liquid in the flexible container and any hydrostatic pressure to which the liquid is subjected in the container. Since the force/pressure against the first pressure indicating transmitter may also depend on the amount of gas pressure in portion 38 above the level of liquid 22 in the container, the gas pressure alone can be measured by second pressure indicating transmitter 30, which may be positioned at or near the top of the container. As described above, the first and second pressure indicating transmitters can be configured to send signals to control system 34, which can subtract the pressure measured by the second pressure indicating transmitter from the pressure measured by the first pressure indicating transmitter, resulting in measurement of the pressure exerted by only the liquid head in the container. This value can be converted to weight and/or volume through formulas relating the mass and density of the liquid, as well the volume of the container, as would be apparent to those skilled in the art.

The second pressure or force indicating transmitter may be connected to the flexible container via hose 36 and, optionally, via a gauge guard 37, which can isolate the pressure indicating transmitter from the contents of container 18 while allowing proper operation of the pressure indicating transmitter. In other embodiments, pressure indicating transmitter 30 can be connected directly to or in fluid communication with the container 18. An exhaust filter 40 connected to the hose may be used for venting an exhaust stream from the container 18.

Also shown in FIG. 1 are an optional inlet port 42 and optional outlet port 46, which can be formed in the flexible container and/or reusable support structure and can facilitate more convenient introduction and removal of a liquid and/or gas from the container. Tubing may be connected to the inlet and/or outlet ports to form delivery and harvest lines, respectively, for introducing and removing liquid from the container. Optionally, the container and/or support structure may include a utility tower 50, which may be provided to facilitate interconnection of one or more devices internal to the container and/or support structure with one or more pumps, controllers, and/or electronics (e.g., sensor electronics, electronic interfaces, and pressurized gas controllers) or other devices. The support structure and/or flexible container may also include one or more ports 54 that can be used for sampling, analyzing (e.g., determining pH and/or amount of dissolved gases in the liquid), or other for other purposes. The support structure may also include one or more site windows 60 for viewing a level of liquid within the flexible container. One or more connections 64 may be positioned near the top of the container or at any other suitable location. Connections 64 may include openings, tubes, and/or valves for adding or withdrawing liquids, gases, and the like from the container, each of which connection may optionally include a flow sensor and/or filter (not shown). The support structure may comprise a plurality of legs 66, optionally with wheels for facilitating transport of the bioreactor system.

It should be understood that not all of the features shown in FIG. 1 need be present in all embodiments of the invention and that the illustrated elements may be otherwise positioned or configured. Also, additional elements may be present in other embodiments.

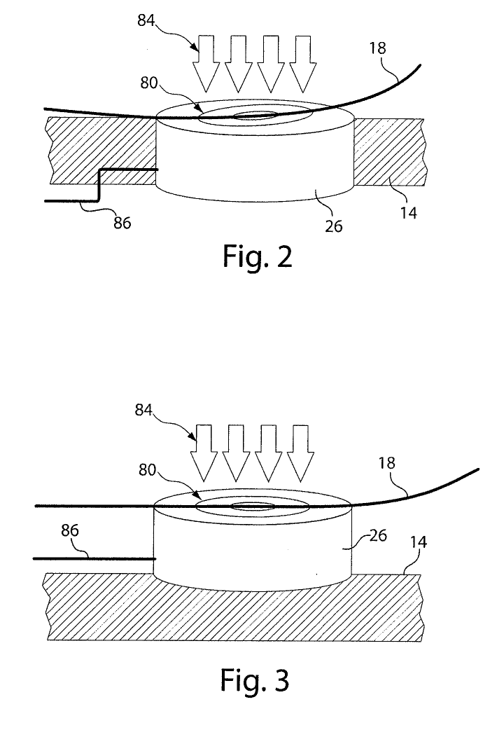

First pressure indicating transmitter 26 may be positioned at any suitable location to obtain an accurate reading of force or pressure exerted by a liquid within the flexible container. In one embodiment, the first pressure indicating transmitter is positioned at or near the bottom of the container, for example, as described above and as shown schematically in FIGS. 2 and 3. The pressure indicating transmitter may be placed substantially horizontally across the bottom of the disposable container, e.g., such that the active sensing surface of the pressure indicating transmitter is essentially coplanar with the bottom wall of the support structure. The pressure indicating transmitter may be mounted on or adjacent to a bottom wall of the support structure as illustrated in FIG. 3, or may be recessed within a load-bearing wall of the reusable structure such that it is positioned flush against the flexible container, as illustrated in FIG. 2. Various pressure indicating sensors can be used in such configurations, including but not limited to ones such as those provided by Rosemount/Emerson Process Management (e.g., the Rosemount 3051 pressure transmitter). When the flexible container contains a liquid, a sensing element of the pressure indicating transmitter, such as diaphragm 80, is deflected by a downward force 84 applied by the liquid above it. The force is transferred through the container material onto the pressure indicating transmitter. The force can, in certain embodiments, create a voltage, which is sent to the control system via cable 86. The control system can convert this voltage into a column height of fluid using a programmed algorithm, which can then be converted into a weight and/or volume measurement of the liquid inside the container, for example using a programmed algorithm that takes into account, for example the known shape and size of the container.

In other embodiments, pressure indicated transmitter 26 can be positioned at or near a side wall of the flexible container such that the flexible container applies a horizontal force against the pressure indicating transmitter. Pressure indicating sensors may also be positioned at other locations relative to the container and the force of the liquid applied against the pressure indicating transmitter can be calibrated to account for its vertical position relative to the bottom of the container to indicate a weight and/or volume of the liquid inside the container.

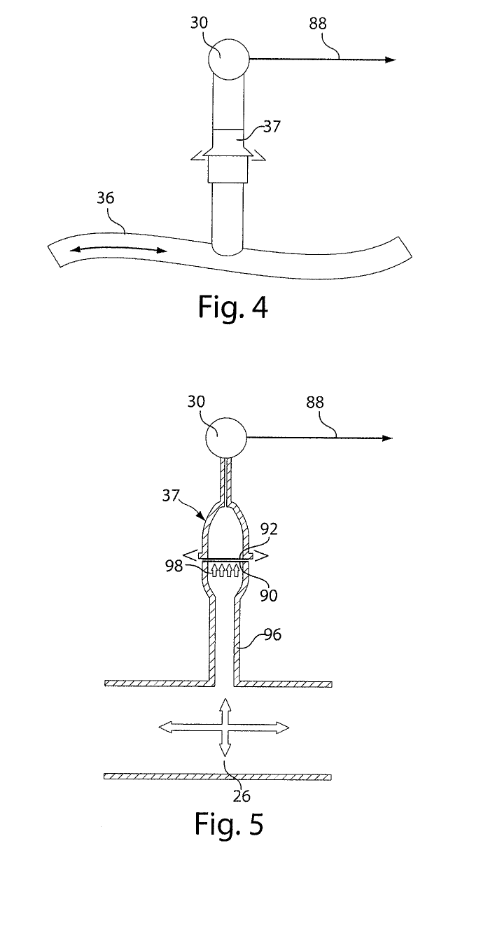

In some embodiments, the flexible container is substantially closed, e.g., the container is substantially sealed from the environment outside of the container except, in certain embodiments, for one or more inlet and/or outlet ports that allow addition to, and/or withdrawal of contents from, the container. The flexible container may be substantially deflated prior to being filled with a liquid, and may begin to inflate as it is filled with liquid. As the container begins to pressurize, the gas pressure above the liquid level, if not vented during filling, exerts a force on the liquid in the container and the combined force of the gas pressure and the liquid head is measured by the first pressure indicating transmitter. Gas pressure may also be created by gas liberation by the reaction taking place in the container (e.g., generated by cellular respiration, fermentation, gas generating chemical reactions, etc.) and/or via introduction of gas to the container during operation (e.g. introduction of air or oxygen to facilitate respiration or carbon dioxide to facilitate photosynthesis). The gas pressure can also be measured directly by a second pressure indicating sensor, which is advantageously positioned above the liquid level. As shown in the embodiment illustrated in FIG. 4, a second pressure indicating transmitter 30 may be operatively associated with the flexible container, for example by being fluidically interconnected to the container via hose 36 and gauge guard 37. The gauge guard can isolate the liquid inside the container from contact with the pressure indicating transmitter, while allowing the pressure indicating transmitter to measure gas pressure inside the container. Gauge guard 37 may be a commercially available article of conventional design and may be formed of any suitable material and, in some instances, may be formed of a flexible material such as silicone so as to provide a conformal seal and permit it to deflect in response to changes in pressure. As shown in the embodiment illustrated in FIG. 4, the gauge guard may be in the form of an integral molded fitting; however, other configurations are also possible. A signal measured by the pressure indicating transmitter can be sent to a control system via cable 88.

FIG. 5 shows pressure indicating transmitter and a cross sectional view of gauge guard 37. As shown in this illustrative embodiment, gauge guard 37 includes a first diaphragm 90 as well as a second diaphragm 92. First diaphragm 90 is attached and sealed to a weldet 96 (e.g., a one piece molded silicone weldet) to allow the sterile boundary of the bag to be maintained. In such an embodiment, gas pressure above the liquid in the container can apply a force in the direction of arrows 98 against the diaphragm to transmit the pressure to pressure indicating transmitter 30, which can detect the pressure and transmit a signal indicative of the measured pressure to the control system.

In some embodiments, first and/or second pressure indicating transmitters 26 and 30 include piezoelectric sensors, which generate a voltage in response to an applied mechanical stress (e.g., force or pressure). In certain embodiments, pressure indicating transmitters/sensors associated with flexible containers described herein (which, in some embodiments, are not be in contact with any fluid inside the flexible container) include those provided by Rosemount/Emerson Process Management (e.g., the Rosemount 3051 pressure transmitter). The first and/or second pressure indicating transmitters can also include sensors that are positioned within the sterile boundary of the bioreactor. In some embodiments, the sensors have a flow-through design and can be used to measure pressure of a liquid or gas flowing through or adjacent a portion of the sensor. For instance, the sensors may be positioned in the gas delivery and harvest lines, which can be used to measure the gas pressure above the liquid level and the liquid force exerted on the bottom of the bag, respectively. It should be understood, however, that other types of pressure sensors can be used (alone or in combination with another sensor or device) to measure a characteristic of a liquid inside the flexible container that is indicative of a weight and/or volume of the liquid.

In some embodiments, one or more pressure indicating transmitters/sensors are disposable. Disposable pressure indicating transmitters/sensors may be associated with the flexible container. For instance, the disposable sensors may be non-integrally and reversibly attached to a tubing (e.g., for measuring the gas pressure above the liquid level and/or the liquid force exerted on the bottom of the bag) and/or positioned between a flexible container and a reusable support structure as described herein. Examples of disposable pressure indicating transmitters/sensors that may be used with a flexible container include those available from PendoTECH and Utah Medical (e.g., the Deltran DPT-100 transducer). Disposable pressure indicating transmitters/sensors may be useful, for example, in certain embodiments involving fluid contact between the pressure indicating transmitters/sensors and the fluid in the flexible container where sterility and/or freedom from contaminants of the pressure indicating transmitters/sensors is important.

As described above, first and/or second pressure indicating transmitters 26 and 30 may be fluidically isolated from contents in the flexible container. In other embodiments, the first and/or second pressure indicating transmitters may also be fluidically isolated from any gas (e.g., a vapor) present inside the flexible container. Accordingly, in such embodiments, the first and/or second pressure indicating transmitters can be reused between subsequent reactions without being cleaned and are prevented from causing any contamination of the contents of the flexible container during use.

The use of pressure indicating transmitters 26 and 30 in certain embodiments allows for real-time monitoring of weight (and/or volume) (e.g., as a function of time) of contents within the flexible container while the system is in operation and while the flexible container is under a positive pressure. In such embodiments, the pressure indicating transmitters may be used to trigger back pressure control devices to maintain desired pressure levels to suit different bioreactor processing requirements. Moreover, because vent filter clogging in disposable bioreactor processes can cause undesirable over-pressurization, which may result in failure of the flexible container, measurements of pressure within the container can be helpful in informing the operator or an automated control system about the status of the vent filters before such an event occurs. For instance, pressure information (e.g., a difference between a first and second pressure as measured by two pressure indicating sensors operatively associated with the flexible container) can be used to automate switching of an exhaust stream from one vent filter 40 to another filter in the case that filter 40 becomes clogged. This can be done without risking integrity of the disposable container by performing a manual filter bypass operation or the system may be configured to perform such a bypass operation automatically upon detection of an overpressure condition.

The weight measurement and back pressure monitoring capabilities of certain embodiments of bioreactor system 10 can also facilitate constant flow processes, such as perfusion processes, to be performed. In some perfusion processes, the bioreactor system provides a growth environment for a cellular matrix wherein the inlet flow of fresh growth medium is balanced by an outlet flow of harvested material (e.g., proteins, enzymes, antibiotics, growth hormones, microbial cells, vitamins, amino acids, and other organic acids). This balance of flows is often correlated with the weight of the cell culture, which can be advantageously measured by the pressure indicating transmitters of certain embodiments of the invention. In certain embodiments, the weight and/or volume of the cell culture can be held constant by adjusting the inlet and outlet flows of fresh growth medium and product according to the weight/volume determinations made by the inventive system. In other embodiments, the pressure indicating transmitters can be used for regulating fed-batch or other types of processes.

Although closed bioreactor systems have primarily been described herein, it would be readily apparent and understood by those skilled in the art that in other embodiments, aspects of the invention can be applied to open bioreactor systems. For instance, a flexible container may be supported by a reusable support structure, both of which are open to atmosphere. In such embodiments, only a single pressure indicating transmitter (e.g., positioned near or at the bottom of the disposable container) may be required for measuring a weight and/or volume of a liquid inside the container.

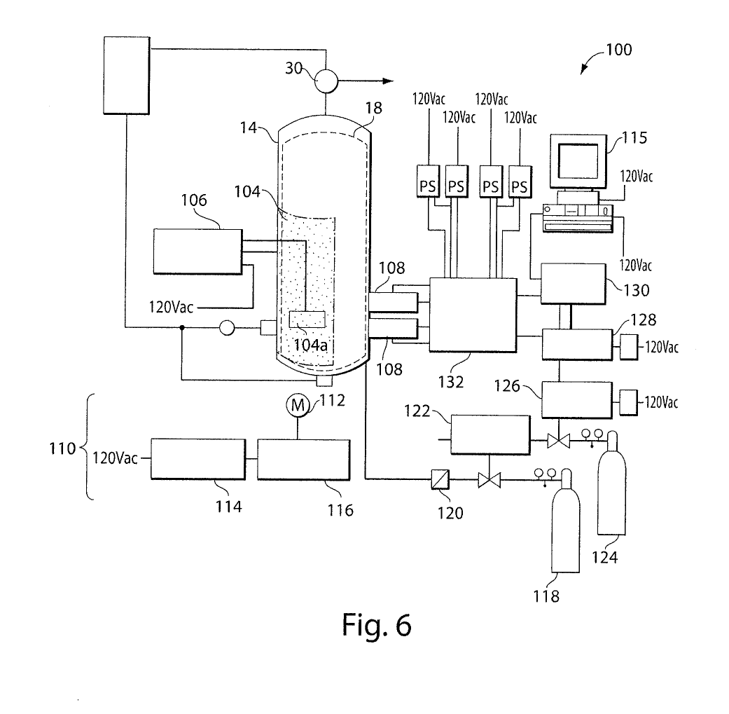

As shown in the exemplary embodiment illustrated in FIG. 6, the flexible container and reusable support structure illustrated in FIG. 1 can be operatively associated with a variety of components as part of an overall bioreactor system 100. Accordingly, the flexible container and/or reusable support structure may include several fittings to facilitate connection to functional component such as filters, sensors, and mixers, as well as connections to lines for providing reagents such as liquid media, gases, and the like. The flexible container and the fittings may be sterilized prior to use so as to provide a “sterile envelope” protecting the contents inside the container from airborne contaminants outside. In some embodiments, the contents inside the container do not contact the reusable support structure and, therefore, the reusable support structure can be reused after carrying out a particular chemical and/or biological reaction without being sterilized, while the flexible container and/or fittings connected to the disposable container can be discarded. In other embodiments, the flexible container, fittings, and/or reusable support structure may be reused (e.g., after cleaning and sterilization).

In some embodiments, the flexible container is a disposable bag that is formed of a suitable flexible material. In some embodiments, the flexible material may be one that is USP Class VI certified, e.g., silicone, polycarbonate, polyethylene, and polypropylene. Non-limiting examples of flexible materials include polymers such as polyethylene (e.g., linear low density polyethylene and ultra low density polyethylene), polypropylene, polyvinylchloride, polyvinylidene chloride, ethylene vinyl acetate, polyvinyl alcohol, silicone rubber, other synthetic rubbers and/or plastics. As noted above, portions of the flexible container may comprise a substantially rigid material such as a rigid polymer (e.g., high density polyethylene), metal, and/or glass (e.g., in areas for supporting fittings, etc.). In other embodiments, the container is formed of substantially rigid materials. All or portions of the container may be optically transparent to allow viewing of contents inside the container.

The flexible container may have any suitable thickness for holding a liquid and may be designed to have a certain resistance to puncturing during operation or while being handled. For instance, the flexible container may have a total thickness of less than or equal to 50 mils, less than or equal to 25 mils, less than or equal to 15 mils, or less than or equal to 10 mils. In some embodiments, the flexible container includes more than one layer of material that may be laminated together or otherwise attached to one another to impart certain properties to the flexible container. For instance, one layer may be formed of a material that is substantially oxygen impermeable. Another layer may be formed of a material to impart strength to the flexible container. Yet another layer may be included to impart chemical resistance to fluid that may be contained in the flexible container. It should be understood that a flexible container may be formed of any suitable combinations of layers and that the invention is not limited in this respect. The flexible container may include, for example, 1 layer, greater than or equal to 3 layers, or greater than equal to 5 layers of material(s). Each layer may have a thickness of, for example, less than or equal to 25 mils, less than or equal to 15 mils, less than or equal to 10 mils, less than or equal to 5 mils, or less than or equal to 3 mils.

The flexible container may have any suitable size for carrying out a chemical and/or biological reaction. For example, the container may have a volume between 1-100 L, 100-200 L, 200-300 L, 300-500 L, 500-750 L, 750-1,000 L, 1,000-2,000 L, or 2,000-5,000 L. Volumes greater than 5,000 L are also possible.

The reusable support structure may be formed of a substantially rigid material. Non-limiting examples of materials that can be used to form the reusable support structure include stainless steel, aluminum, glass, resin-impregnated fiberglass or carbon fiber, polymers (e.g., high-density polyethylene, polyacrylate, polycarbonate, polystyrene, nylon or other polyamides, polyesters, phenolic polymers, and combinations thereof. The materials may be certified for use in the environment in which it is used. For example, non-shedding materials may be used in environments where minimal particulate generation is required.

The reusable support structure may be designed to have a height and diameter similar to standard stainless steel bioreactors. The design may also be scaleable down to small volume bench bioreactor systems. Accordingly, the reusable support structure may have any suitable volume for carrying out a desired chemical and/or biological reaction. In many instances, the reusable support structure has a volume substantially similar to that of the flexible container. For instance, a single reusable support structure may be used to support and contain and single flexible container having a substantially similar volume. In other cases, however, a reusable support structure is used to contain more than one container. The reusable support structure may have a volume between 1-100 L, 100-200 L, 200-300 L, 300-500 L, 500-750 L, 750-1,000 L, 1,000-2,000 L, or 2,000-5,000 L. Volumes greater than 5,000 L are also possible.

As shown in the embodiment illustrated in FIG. 6, the flexible container 18 may be operatively associated with a temperature controller 106 which may be, for example, a heat exchanger, a closed loop water jacket, an electric heating blanket, or a Peltier heater. Other heaters for heating a liquid inside a container are known to those of ordinary skill in the art and can also be used in combination with flexible container 18. The heater may also include a thermocouple or a resistance temperature detector (RTD) for sensing a temperature of the contents inside the container. The thermocouple may be operatively connected to the temperature controller to control temperature of the contents in the container. Optionally, a heat-conducting material may be embedded in the surface of the flexible container to provide a heat transfer surface to overcome the insulating effect of the material used to form other portions of the container.

Cooling may also be provided by a closed loop water jacket cooling system, a cooling system mounted on the bioreactor, or by standard heat exchange through a cover/jacket on the reusable support structure (e.g., the heat blanket or a packaged dual unit which provides heating and cooling may a component of a device configured for both heating/cooling but may also be separate from a cooling jacket). Cooling may also be provided by means of Peltier coolers. For example, a Peltier cooler may be applied to an exhaust line to condense gas in the exhaust air to help prevent an exhaust filter from wetting out.

The container 18 may also include various sensors and/or probes 108 for controlling and/or monitoring one or more process parameters inside the disposable container such as, for example, temperature, pressure, pH, dissolved oxygen (DO), dissolved carbon dioxide (DCO₂), mixing rate, and gas flow rate. In some embodiments, process control may be achieved in ways which do not compromise the sterile barrier established by a disposable container. For example, gas flow may be monitored and/or controlled by a rotameter or a mass flow meter upstream of an inlet air filter. In another embodiment, disposable optical probes may be designed to use “patches” of material containing an indicator dye which can be mounted on the inner surface of the disposable container and read through the wall of the disposable container via a window in the reusable support structure. For example, dissolved oxygen, pH, and/or CO₂ each may be monitored and controlled by an optical patch and sensor mounted on, e.g., a gamma-irradiatable, biocompatible polymer which, can be sealed to, embedded in, or otherwise attached to the surface of the container.

In some embodiments, sensors and/or probes may be connected to a sensor electronics module 132, the output of which can be sent to a terminal board 130 and/or a relay box 128. Results of the sensing operations may be input into a computer-implemented control system 115 (e.g., a computer) for calculation and control of various parameters (e.g., weight/volume measurement as provided according to the invention) and for display and user interface. Such a control system may also include a combination of electronic, mechanical, and/or pneumatic systems to control heat, air, and/or liquid delivered to or withdrawn from the disposable container as required to stabilize or control the environmental parameters of the process operation. An example of this is a valve, which may be controlled to switch flow to a new vent filter in the event of a bag pressure sensor signaling a high pressure condition. It should be appreciated that the control system may perform other functions and the invention is not limited to having any particular function or set of functions.

The one or more control systems can be implemented in numerous ways, such as with dedicated hardware and/or firmware, using a processor that is programmed using microcode or software to perform the functions recited above or any suitable combination of the foregoing. A control system may control one or more operations of a single bioreactor, or of multiple (separate or interconnected) bioreactors.

Each of systems described herein and illustrated in FIG. 6, and components thereof, may be implemented using any of a variety of technologies, including software (e.g., C, C#, C++, Java, or a combination thereof), hardware (e.g., one or more application-specific integrated circuits), firmware (e.g., electrically-programmed memory) or any combination thereof.

Various embodiments according to the invention may be implemented on one or more computer systems. These computer systems, may be, for example, general-purpose computers such as those based on Intel PENTIUM-type and XScale-type processors, Motorola PowerPC, Motorola DragonBall, IBM HPC, Sun UltraSPARC, Hewlett-Packard PA-RISC processors, any of a variety of processors available from Advanced Micro Devices (AMD) or any other type of processor. It should be appreciated that one or more of any type of computer system may be used to implement various embodiments of the invention. The computer system may include specially-programmed, special-purpose hardware, for example, an application-specific integrated circuit (ASIC). Aspects of the invention may be implemented in software, hardware or firmware, or any combination thereof. Further, such methods, acts, systems, system elements and components thereof may be implemented as part of the computer system described above or as an independent component.

The flexible container 18 may also be connected to one or more sources of gases 118 and 124 such as air, oxygen, and/or carbon dioxide (compressed or pumped). Such gases may be used to provide suitable growth and/or reaction conditions for producing a product inside the container. The gases may also be used to provide sparging to the contents inside the container for mixing purposes. For instance, in certain embodiments employing sparged reactors, bubble size and distribution can be controlled by passing an inlet gas stream through a porous surface prior to being added to the container. Additionally, the sparging surface may be used as a cell separation device by alternating pressurization and depressurization (or application of vacuum) on the exterior surface of the porous surface, or by any other suitable method. The inlet gases may optionally pass through filter 120 and/or a flow meter and/or valve 122, which may be controlled by controller system 115, prior to entering the container. Valve 122 may be a pneumatic actuator (actuated by, e.g., compressed air/carbon dioxide or other gas 124), which may be controlled by a solenoid valve 126. These solenoid valves may be controlled by a relay 128 connected to terminal board 130, which is connected to the controller system 115. The terminal board may comprise, for example, a PCI terminal board, or a USB/parallel, or fire port terminal board of connection.

The flexible container may also include a mixing system 110 for mixing contents inside the container. Various methods for mixing fluids can be implemented in the container. For instance, mixers based on magnetic actuation, sparging, and air-lift can be used. In one particular embodiment, mixing systems such as the ones disclosed in U.S. Patent Publication No. 2005/0272146, by Hodge et al., entitled “Disposable Bioreactor Systems and Methods”, which is incorporated herein by reference in its entirety, are used with embodiments described herein. For example, the mixing system may include a motor 112, e.g., for driving an impeller (or other component used for mixing) positioned inside the container, a power conditioner 114, and/or a motor controller 116.

In some embodiments, mixing systems, flexible bags, support structures, or other components and/or systems, such as those described in the following references, are combined with embodiments described herein: U.S. Provisional Patent Application Ser. No. 60/903,977, filed Feb. 28, 2007, entitled “Weight Measurements of Liquids in Flexible Containers,” by P. A. Mitchell, et al.; U.S. patent application Ser. No. 11/147,124, filed Jun. 6, 2005, entitled “Disposable Bioreactor Systems and Methods,” by G. Hodge, et al., published as U.S. Patent Application Publication No. 2005/0272146 on Dec. 8, 2005; International Patent Application No. PCT/US2005/020083, filed Jun. 6, 2005, entitled “Disposable Bioreactor Systems and Methods,” by G. Hodge, et al., published as WO 2005/118771 on Dec. 15, 2005; International Patent Application No. PCT/US2005/002985, filed Feb. 3, 2005, entitled “System and Method for Manufacturing,” by G. Hodge, et al., published as WO 2005/076093 on Aug. 18, 2005; U.S. patent application Ser. No. 11/818,901, filed Jun. 15, 2007, entitled, “Gas Delivery Configurations, Foam Control Systems, and Bag Molding Methods and Articles for Collapsible Bag Vessels and Bioreactors”; U.S. application Ser. No. 11/879,033, filed Jul. 13, 2007, entitled “Environmental Containment Systems”; U.S. Application Ser. No. 60/962,671, filed Jul. 30, 2007, entitled, “Continuous Perfusion Bioreactor System”; U.S. Application Ser. No. 60/903,977, filed Feb. 28, 2007, entitled “Weight Measurements of Liquids in Flexible Containers”; U.S. patent application Ser. No. 12/011,492, filed on Jan. 25, 2008, entitled, “Information Acquisition and Management Systems and Methods in Bioreactor Systems and Manufacturing Facilities”; and U.S. patent application Ser. No. 12/011,493, filed on Jan. 25, 2008, entitled, “Bag Wrinkle Remover, Leak Detection Systems, and Electromagnetic Agitation for Liquid Containment Systems”, each of which is incorporated by reference in its entirety for all purposes.

The following examples are intended to illustrate certain embodiments of the present invention, but are not to be construed as limiting and do not exemplify the full scope of the invention.

### EXAMPLE 1

This example describes a configuration of a bioreactor including a disposable, flexible container with pressure indicating sensors that can be used for determining a weight and/or volume of a fluid in the container.

A flexible, disposable bag was installed in a reusable support structure with pressure indicating sensors installed within the sparge air supply line (e.g., at the top of the flexible container), and within a harvest line (e.g., a tubing connected to an outlet port at the bottom of the flexible container). The disposable, flexible bag was formed of a plastic sheet welded together in a designed configuration to result in an enclosed bag design. Hose barb ports were welded on the bag and hoses were installed on these hose barb ports. The number of ports used was dependent on the configuration of the bag necessary to operate a specific bioreactor process. The sensors had a flow-through design to measure pressure of a liquid or gas flowing through them (e.g., a Deltran DPT-100 transducer or other suitable sensor).

The pressure sensors required a voltage excitation, and produced a return voltage based upon the pressure/force exerted upon them. The pressure sensors were excited through a control system and the return signal was connected back to the control system for processing. The signal received from the sensor was processed and displayed on the control system screen. Pressure units tested were measured in psig, but can be converted to weight and/or volume measurements.

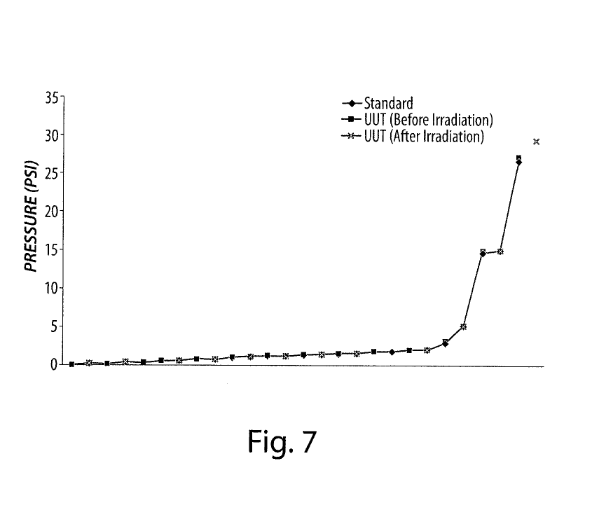

In one experiment, a disposable pressure sensor was attached to the sparge air supply line at the top of the flexible bag and the bag, initially deflated, was inflated from 0 to 2 psig at 0.1 psig increments. The pressure sensor was used to measure pressure inside the bag at each of the increments. A standard sensor (a NIST certified pressure calibrator device), used as a reference, was attached to the same sparge air supply line to independently measure the pressure. FIG. 7 shows the results from the experiment. The plot shows pressure measured by the pressure sensors as the flexible container was inflated. The results indicated that the response from the disposable sensor was similar to that of the NIST certified pressure calibrator device.

The disposable pressure sensor was removed from the bag and irradiated with gamma irradiation to test the sensors' resistance to gamma irradiation. The sensors was then reconnected to the bag and a duplication of the above pressure testing was performed. Similar measurements were obtained to that of the first experiment, which indicated that operation of the pressure sensors was not affected by irradiation. The accuracy of both experiments was measured to 0.01 psig. Similar testing was performed in the manner described above using the sensor installed at the bottom of the bioreactor bag at the harvest line.

### EXAMPLE 2

This prophetic example describes a configuration of a bioreactor including a disposable, flexible container with a first pressure indicating sensor positioned near the bottom of the flexible container that is not in fluid contact with any fluid in the flexible container, and a second pressure indicating sensor positioned near the top of the flexible container and operatively associated with the container via a sparge air supply line. The pressure indicating sensors are used for determining a weight and/or volume of a fluid in the container.

A disposable, flexible container for containing materials for performing biological and/or chemical reaction is supported by a reusable support structure. A first, non-flow through pressure indicating sensor is positioned between the bottom of the flexible container and a wall of the reusable support structure as shown in FIG. 2 and/or 3 (e.g., the pressure indicating sensor is not attached to harvest line). The pressure indicating sensor may be mounted flush against the bottom of the flexible container and is used to measure the total downward force/pressure within the container, including the force/pressure due to the weight of the liquid in the container and also any gas pressure present above the liquid. This lower pressure indicating sensor may be non-disposable since it is not in fluid contact with the fluid inside the flexible container. Various pressure indicating sensors can be used, such as ones provided by Rosemount/Emerson Process Management (e.g., the Rosemount 3051 pressure transmitter or other suitable sensor). A second pressure indicating sensor (e.g., a Deltran DPT-100 transducer or other suitable sensor) is positioned at the top of the disposable, flexible container within the sparge air supply line to measure the gas pressure above the liquid in the container. The first and second pressure indicating sensors are connected to a control system that receives and processes signals from the sensors, and weight and/or volume of a liquid in the flexible container is determined (e.g., as described in Example 1 and in the Detailed Description).

The flexible container is filled with a liquid containing reagents for performing a biological and/or chemical reaction. As the reaction proceeds, the pressure indicating sensors are excited through the control system and return signals are connected back to the control system for processing. The signals received from the sensors are processed using formulas relating the mass and density of the liquid and the volume of the flexible container to obtain a weight and/or volume of the liquid in the container. Similar measurements are obtained as a function of time and as product from the reaction is removed from the flexible container, and/or additional reagents are introduced into the container. In some cases, the measurements will be obtained continuously.

### EXAMPLE 3

This prophetic example describes a configuration of a bioreactor including a disposable, flexible container with a first pressure indicating sensor positioned near the bottom of the flexible container and operatively associated with the container via a harvest line, and a second pressure indicating sensor positioned near the top of the flexible container and operatively associated with the container via a sparge air supply line. The pressure indicating sensors are used for determining a weight and/or volume of a fluid in the container.

Pressure indicating sensors are interconnected with a disposable, flexible container in the manner described in Example 1. A first pressure indicating sensor is positioned at the bottom of the disposable, flexible container in the harvest line and is used to measure the total downward force/pressure within the container, including the force/pressure due to the weight of the liquid in the container and also any gas pressure present above the liquid. A second pressure indicating sensor is positioned at the top of the disposable, flexible container within the sparge air supply line to measure the gas pressure above the liquid. The first and second pressure indicating sensors have flow-through designs and are in contact with fluid in the flexible container. Accordingly, the pressure indicating sensors may be disposable sensors (e.g., a Deltran DPT-100 transducer or other suitable sensor). Non-disposable sensors can also be used. The two pressure indicating sensors are connected to a control system that receives and processes signals from the sensors, and weight and/or volume of a liquid in the flexible container is determined, as described above.

### EXAMPLE 4

This prophetic example describes one method of calculating a weight and/or volume of a liquid in a flexible container using a first lower pressure indicating sensor positioned at the bottom of the flexible container and a second upper pressure indicating sensor positioned at the top of the flexible container (e.g., as described in Example 2). The flexible container in the present example is contained within and has a shape and a volume substantially the same as a circular cylinder—shaped reusable support structure.

On the controller screen, the user will input the specific gravity of the culture (if known weight measurements are desired). The control system will receive the raw signals from the first and second pressure indicating sensors. This control system (or the sensors themselves) will convert the raw voltage signals from each sensor into, for example, pressure readings, for example in units of mmHg, mm H₂O, or psig, utilizing the same units for each sensor. The control system will then subtract the upper sensor value from the lower sensor value. The control system will then convert this difference into a mass and/or volume of the liquid contained in the flexible container by taking into account the geometry (size and shape) of the container and specific gravity of the liquid. For example, the following calculations may be performed for a particular measurement data point:

In a system in which pressure difference is measured in units of inches of water, the measurement of pressure of the first lower pressure transducer measures is 33.84 inches of water. The top transducer, measuring the pressure of the air in the headspace in the flexible container above the liquid, measures a pressure in the headspace of 13.84 inches of water. To obtain the pressure directly resulting from the weight of the liquid in the flexible container, the control system can subtract 13.84 inches of water from 33.84 inches of water to get 20 inches of water.

To convert to volume and weight, the internal diameter of the reusable support structure (and disposable container) is used. For a 200 L reusable support structure having a diameter of 25.5625 inches, the volume will be 1 in(π(12.781 in)²×0.0164 liter/cubic in, or 8.41 Liters for each inch of height of the reusable support structure. For the measured 20 inches of water difference in pressure, this translates to 20×8.41=168.20 liters of volume in water equivalent. The total weight of the process fluid can be obtained by multiplying this volume by the density of water (1 kg/liter): 168.20 liters (1 kg/liter)=168.20 kg. To obtain the volume of the actual process fluid, the water equivalent volume value can be divided by the specific gravity of the process fluid. This value obtained will be linear for the response from the pressure sensors. The control system can be scaled to output or display the correct volume given the response of the sensors. This system can also be coupled with on-line OD sensors in the culture fluid, which can be used to determine the on-line optical density of the fluid. This function would allow continuous specific gravity inputs from the OD sensor to be used in the weight calculation to gain additional accuracy of weight measurement in a dynamic cell culture process.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

What is claimed is:

1. A bioreactor system comprising:
a flexible container for housing a liquid, the container;
a reusable support structure for surrounding and containing the flexible container;
a first pressure or force indicating sensor operatively associated with the flexible container but not in fluid contact with any fluid in the flexible container, the pressure or force indicating sensor configured to measure a parameter indicative of a first pressure in the flexible container;
a second pressure or force indicating sensor operatively associated with the flexible container, the pressure or force indicating sensor configured to measure a parameter indicative of a second pressure in the flexible container; and
a control system configured to receive input from the first and second pressure or force indicating sensors indicative of the first and second pressures and to calculate a difference between the first and second pressures.

2. A bioreactor as in claim 1, further comprising determining a volume or a weight of a liquid in the flexible container using the difference between the first and second pressures.

3. A bioreactor system as in claim 1, wherein the flexible container is configured to be removable from the reusable support structure.

4. A bioreactor system as in claim 3, wherein the flexible container is in the form of a disposable bag.

5. A bioreactor system as in claim 4, wherein the disposable bag is sterile prior to use.

6. A bioreactor system as in claim 1, wherein the flexible container includes at least one inlet port and at least one outlet port.

7. A bioreactor system as in claim 1, wherein the control system is configured to receive signals from the first and second pressure or force indicating sensors, the signals comprising the input indicative of the first and second pressures.

8. A bioreactor system as in claim 1, wherein the first and/or second pressure or force indicating sensors comprises a measuring cell for sensing a force or pressure and an electronic circuit to process a signal from the measuring cell.

9. A bioreactor system as in claim 1, wherein the first and/or second pressure or force indicating sensors comprises a piezoelectric sensor.

10. A bioreactor system as in claim 1, wherein the first pressure or force indicating sensor senses a force applied to the flexible container by a liquid in the flexible container.

11. A bioreactor system as in claim 1, wherein the first pressure or force indicating sensor is positioned at a bottom of the flexible container.

12. A bioreactor system as in claim 1, wherein the first pressure or force indicating sensor is positioned at an inner surface of the reusable support structure and the inner surface is in contact with the flexible container.

13. A bioreactor system as in claim 1, wherein the first pressure or force indicating sensor is mounted flush at a bottom surface of the reusable support structure.

14. A bioreactor system as in claim 1, wherein the first and/or second pressure or force indicating sensor is not in contact with a liquid in the flexible container.

15. A bioreactor system as in claim 1, wherein the first and/or second pressure or force indicating sensor is not in contact with a gas in the flexible container.

16. A bioreactor system as in claim 1, wherein the flexible container forms a closed system.

17. A bioreactor system as in claim 1, wherein the flexible container forms an open system.

18. A bioreactor system as in claim 4, wherein the disposable bag is substantially deflated prior to being filled with a liquid.

19. A bioreactor system as in claim 1, further comprising a gauge guard positioned between the second pressure or force indicating sensor and the flexible container.

20. A bioreactor system as in claim 1, further comprising a mixer.

21. A bioreactor system as in claim 20, wherein the mixer comprises an impeller positioned inside the flexible container.

22. A method comprising determining a weight and/or volume of the liquid in the flexible container of claim 1.

23. A method as in claim 22, further comprising determining a status of a vent filter operatively associated with the bioreactor system of claim A using one or more measurements from the first and/or second pressure or force indicating sensors.

24. A bioreactor system comprising:
a flexible container for housing a liquid in the container;
a reusable support structure for surrounding and containing the flexible container;
a first pressure or force indicating sensor operatively associated with the flexible container and configured to measure a parameter indicative of a first pressure in the flexible container;
a second pressure or force indicating sensor operatively associated with the flexible container and configured to measure a parameter indicative of a second pressure in the flexible container; and
a control system configured to receive input from the first and second pressure or force indicating sensors indicative of the first and second pressures and to calculate a difference between the first and second pressures.

25. A bioreactor as in claim 24, further comprising determining a volume or a weight of a liquid in the flexible container using the difference between the first and second pressures.

26. A bioreactor system comprising:
a flexible container for housing a liquid in the container;
at least one pressure or force indicating sensor operatively associated with the flexible container but not in contact with any fluid in the flexible container, the pressure or force indicating sensor configured to measure a parameter indicative of a pressure in the flexible container, and
a control system configured to receive input from the at least one pressure or force indicating sensor indicative of the pressure, the control system configured to determine from the input a volume or a weight of a liquid in the flexible container.
